(19) United States
(12) Patent Application Publication
(10) Pub. No.: US 2020/0268017 A1
(43) Pub. Date: Aug. 27, 2020

(54) PROBIOTIC COMPOSITIONS AND METHODS

(71) Applicant: CORNELL UNIVERSITY, Ithaca, NY (US)

(72) Inventor: Rodrigo Carvalho Bicalho, Dryden, NY (US)

(21) Appl. No.: 16/630,675

(22) PCT Filed: Jun. 20, 2018
(86) PCT No.: PCT/US2018/038468

§ 371 Date: Jan. 13, 2020

Related U.S. Application Data
(60) Provisional application No. 62/522,328, filed on Jun. 20, 2017.

(51) Int. Cl. A23K 10/16; A23K 50/10; A61K 35/741

(52) U.S. Cl. CPC A23K 10/16; A61K 35/741; A23K 50/10

(57) ABSTRACT

The present invention relates to probiotic compositions and methods of using such compositions. In particular, the present invention provides methods of using Faecalibacterium spp. and composition derived from culture of Faecalibacterium spp. to prevent or decrease growth of other microorganisms, particularly pathogenic organisms.

### CROSS REFERENCE TO RELATED APPLICATION

This application claims priority to and the benefit of U.S. Provisional Application No. 62/522,328, filed Jun. 20, 2017, which is hereby incorporated by reference in its entirety.

### FIELD OF INVENTION

The present invention relates to probiotic compositions and methods of using such compositions. In particular, the present invention provides methods of using Faecalibacterium spp. and composition derived from culture of Faecalibacterium spp. to prevent or decrease growth of other microorganisms, particularly pathogenic organisms.

### BACKGROUND OF THE INVENTION

Gut microbiota is known to have a role in shaping key aspects of postnatal life, such as the development of the immune system (Mazmanian et al., (2005) Cell 122(1): 107-118; Peterson et al., (2007) Cell Host Microbe 2(5): 328-339), and influencing the host's physiology, including energy balance. Transplanting the gut microbiota from normal mice into germ-free recipients increased their body fat without any increase in food consumption, raising the possibility that the composition of the microbial community in the gut affects the amount of energy extracted from the diet (Backhed et al., (2004) Proc Natl Acad Sci USA 101(44): 15718-15723). There is at least one type of obesity-associated gut microbiome characterised by higher relative abundance of Firmicutes or a higher Firmicutes to Bacteroidetes ratio (Ley et al., (2005) Proc Natl Acad Sci USA 102(31): 11070-11075; Tumbaugh et al., (2006) Nature 444(7122): 1027-1031). The role of intestinal microbiota in disease has also been shown. Gut microbes serve their host by functioning as a key interface with the environment; for example, they can protect the host organism from pathogens that cause infectious diarrhea. A decreased diversity of fecal microbiota and specifically a reduced diversity of Firmicutes in Crohn's disease patients has been reported (Manichanh et al., (2006) Gut 55(2): 205-211), while it was recently shown that Faecalibacterium prausnitzii displays anti-inflammatory action and can potentially be used for the treatment of this disease (Sokol et al., (2008) Proc Natl Acad Sci USA 105(43): 16731-16736).

### SUMMARY OF THE INVENTION

The present invention relates to probiotic compositions and methods of using such compositions. In particular, the present invention provides methods of using Faecalibacterium spp. and composition derived from culture of Faecalibacterium spp. to prevent or decrease growth of other microorganisms, particularly pathogenic organisms.

Accordingly, in some embodiments, the present invention provides methods of preventing or inhibiting growth of a target microorganism in a subject in need thereof comprising administering to the subject an effective amount of a Faecalibacterium spp. composition. In some embodiments, the present invention provides for the use of a Faecalibacterium spp. composition to prevent or inhibit growth of a target microorganism. In preferred embodiments, the Faecalibacterium spp. strain or strains utilized exhibit or have antimicrobial activity as assayed by an in vitro assay.

In some embodiments, the Faecalibacterium spp. is Faecalibacterium prausnitzii. In some embodiments, the Faecalibacterium prausnitzii is strain 24, 30, 266 or 267. In some embodiments, the Faecalibacterium spp. composition comprises an effective amount of live Faecalibacterium spp. In some embodiments, the Faecalibacterium spp. composition comprises an effective amount of dead or inactivated Faecalibacterium spp. In some embodiments, the Faecalibacterium spp. composition is formulated powder, bolus, gel, liquid drench, capsule, or paste. In some embodiments, the Faecalibacterium spp. composition further comprises a bulking agent. In some embodiments, the bulking agent is selected from the group consisting of milk powder, skim milk powder, corn starch, corn meal, and soybean meal.

In some embodiments, the Faecalibacterium spp. composition is a Faecalibacterium spp. culture supernatant from a culture of the Faecalibacterium spp. In some embodiments, the Faecalibacterium spp. composition is formulated powder, bolus, gel, liquid drench, capsule, or paste. In some embodiments, the Faecalibacterium spp. composition further comprises a bulking agent. In some embodiments, the bulking agent is selected from the group consisting of milk powder, skim milk powder, corn starch, corn meal, and soybean meal.

In some embodiments, the composition is coadministered with at least a second probiotic organism selected from the group consisting of Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, and combinations thereof.

In some embodiments, the composition is formulated with an additional additive selected from the group consisting of an energy substrate, a mineral, a vitamin, and combinations thereof.

In some embodiments, the target microorganism is a pathogenic microorganism. In some embodiments, the target microorganism is selected from the group consisting of Escherichia coli, Klebsiella pneumoniae and Staphylococcus aureus.

In some embodiments, the animal is a domestic animal. In some embodiments, the domestic animal is selected from the group consisting of cattle, sheep, swine, and horses. In some embodiments, the animal is a calf.

In some embodiments, the amount is effective to kill the target microorganism. In some embodiments, the amount is effective to reduce the amount of the microorganism in the organism by at least 50% as compared to the amount of microorganism present prior to administration. In some embodiments, the amount is effective to treat or prevent an infection by the target microorganism.

### DESCRIPTION OF THE FIGURES

FIG. 1. The effect of F. prausnitzii supernatant on the growth of E. coli ATCC 31616. The supernatant of F. prausnitzii strains were collected after 24 h incubation by using the same culture medium. The different proportion of SN (4%, 8%, 16%, 32%, 64%, 100%) were added into a 96-well plate with a final volume of 300 μl supplemented by LB broth, respectively. The OD₆₀₀ of samples were detected every half an hour until 24 h incubation by using a microwell plate reader (BioTek). The values are expressed as the mean±SEM after three independent experiments.

FIG. 2. The effect of F. prausnitzii supernatant on the growth of E. coli ATCC 25922. The supernatant of F. prausnitzii strains were collected after 24 h incubation by using the same culture medium. The different proportion of SN (8%, 16%, 32%, 64%, 100%) were added into a 96-well plate with a final volume of 300 μl supplemented by LB broth, respectively. The OD₆₀₀ of samples were detected every half an hour by using a microwell plate reader (BioTek). The values are expressed as the mean±SEM after three independent experiments.

FIG. 3. The effect of F. prausnitzii supernatant on the growth of Klebsiella pneumoniae strain 22326. The supernatant of F. prausnitzii strains were collected after 24 h incubation by using the same culture medium. The different proportion of SN (8%, 16%, 32%, 64%, 100%) were added into a 96-well plate with a final volume of 300 μl supplemented by LB broth, respectively. The OD₆₀₀ of samples were detected every half an hour by using a microwell plate reader (BioTek). The values are expressed as the mean±SEM after three independent experiments.

FIG. 4. The effect of F. prausnitzii supernatant on the growth of Staphylococcus aureus ATCC 27708. The supernatant of F. prausnitzii strains were collected after 24 h incubation by using the same culture medium. The different proportion of SN (8%, 16%, 32%, 64%, 100%) were added into a 96-well plate with a final volume of 300 μl supplemented by LB broth, respectively. The OD₆₀₀ of samples were detected every half an hour by using a microwell plate reader (BioTek). The values are expressed as the mean±SEM after three independent experiments.

FIG. 5. The effect of F. prausnitzii supernatant on the growth of Methicillin-resistant Staphylococcus aureus (MRSA) 4454. The supernatant of F. prausnitzii strains were collected after 24 h incubation by using the same culture medium. The different proportion of SN (8%, 16%, 32%, 64%, 100%) were added into a 96-well plate with a final volume of 300 μl supplemented by LB broth, respectively. The OD₆₀₀ of samples were detected every half an hour by using a microwell plate reader (BioTek). The values are expressed as the mean±SEM after three independent experiments.

FIG. 6. The F. prausnitzii supernatant cause the reduction of number of E. coli ATCC 31616. The supernatant of F. prausnitzii strains were collected after 24 h incubation by using the same culture medium. The cell of E. coli ATCC 31616 were recovered after it reached the stationary phase (16 h incubation). The different proportion of SN (4%, 8%, 16%, 32%, 64%) were added into a 96-well plate with a final volume of 300 μl supplemented by E. coli cells, respectively. The OD₆₀₀ of samples were detected every half an hour by using a microwell plate reader (BioTek). The values are expressed as the mean±SEM after three independent experiments.

FIG. 7. CFU counting of different bacteria after co-incubation with the supernatant of F. prausnitzii strains 24. The cell of various bacteria were recovered after it reached the stationary phase. Then the cell of bacteria was co-incubated with a 50% volume of supernatant of F. prausnitzii strains 24. The mixtures of supernatant and E. coli were taken at 60 min and serially diluted before CFU counting. The values are expressed as the mean±SD after three independent experiments.

### DEFINITIONS

To facilitate an understanding of the present invention, a number of terms and phrases are defined below:

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used herein, the term “in vitro” refers to an artificial environment and to processes or reactions that occur within an artificial environment. In vitro environments can consist of, but are not limited to, test tubes, microtiter plates, and the like. The term “in vivo” refers to the natural environment (e.g., an animal or a cell) and to processes or reactions that occur within a natural environment.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

Mammals are defined herein as all animals (e.g., human or non-human animals) that have mammary glands and produce milk.

As used herein, a “dairy animal” refers to a milk producing non-human mammal that is larger than a laboratory rodent (e.g., a mouse). In preferred embodiments, the dairy animals produce large volumes of milk and have long lactating periods (e.g., cows or goats).

A “subject” is an animal such as vertebrate, preferably a domestic animal or a mammal. Mammals are understood to include, but are not limited to, murines, simians, humans, bovines, cervids, equines, porcines, canines, felines etc.

An “effective amount” is an amount sufficient to effect beneficial or desired results. An effective amount can be administered in one or more administrations,

“Co-administration” refers to administration of more than one agent or therapy to a subject. Co-administration may be concurrent or, alternatively, the chemical compounds described herein may be administered in advance of or following the administration of the other agent(s). One skilled in the art can readily determine the appropriate dosage for co-administration. When co-administered with another therapeutic agent, both the agents may be used at lower dosages. Thus, co-administration is especially desirable where the claimed compounds are used to lower the requisite dosage of known toxic agents.

As used herein, the term “toxic” refers to any detrimental or harmful effects on a cell or tissue.

A “pharmaceutical composition” is intended to include the combination of an active agent with a carrier, inert or active, making the composition suitable for diagnostic or therapeutic use in vivo or ex vivo.

As used herein, the term “pharmaceutically acceptable carrier” encompasses any of the standard pharmaceutical carriers, such as a phosphate buffered saline solution, water, and an emulsion, such as an oil/water or water/oil emulsion, and various types of wetting agents.

The compositions also can include stabilizers and preservatives. For examples of carriers, stabilizers and adjuvants see Martin, Remington's Pharmaceutical Sciences, 15th Ed., Mack Publ. Co., Easton, Pa. (1975).

“Pharmaceutically acceptable salt” as used herein, relates to any pharmaceutically acceptable salt (acid or base) of a compound of the present invention, which, upon administration to a recipient, is capable of providing a compound of this invention or an active metabolite or residue thereof. As is known to those of skill in the art, “salts” of the compounds of the present invention may be derived from inorganic or organic acids and bases. Examples of acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds of the invention and their pharmaceutically acceptable acid.

As used herein, the term “nutraceutical,” refers to a food substance or part of a food, which includes a probiotic bacterium. Nutraceuticals can provide medical or health benefits, including the prevention, treatment, or cure of a disorder.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

### DETAILED DESCRIPTION OF THE INVENTION

The present invention relates to probiotic compositions and methods of using such compositions. In particular, the present invention provides methods of using Faecalibacterium spp. and composition derived from culture of Faecalibacterium spp. to prevent or decrease growth of other microorganisms, particularly pathogenic organisms.

Changes in the microbial community are observed in individuals with intestinal inflammatory disorders. These changes are often accompanied by a decrease of obligate anaerobic bacteria, whereas the relative abundance of facultative anaerobic Enterobacteriaceae increases (Winter, et al., 2013). Inflammatory bowel diseases (IBD), including Crohn's disease (CD) and ulcerative colitis (UC), are multifactorial ailments characterized by intestinal inflammation (Huang, et al., 2016). Faecalibacterium prausnitzii, is an extremely oxygen sensitive commensal butyrate producer bacterium belonging to the Clostridium leptum group of the gut microbiota, recognized as a biomarker of intestinal health (Miquel, et al., 2013). F. prausnitzii is an anti-inflammatory commensal bacterium identified on the basis of human clinical data (Hornef, et al., 2016). F. prausnitzii produces high amounts of butyrate, which has well known anti-inflammatory effects (Segain, et al., 2000). A previous study (Winter, et al., 2013) showed that nitrate generated as a by-product of the inflammatory response conferred a growth advantage to the commensal bacterium E. coli in the large intestine of mice. However, the mechanisms underlying its beneficial effects on human and animal health are still unknown.

The reduction of F. prausnitzii was associated with higher risk postoperative recurrence of ileal Crohn Disease (CD) (Sokol, et al., 2008). A significantly lower proportion of F. prausnitzii at the time of surgery, consistently associated with endoscopic relapse was observed. In vitro peripheral blood mononuclear cell stimulation by F prausnitzii led to significantly lower IL-12 and IFN-γ production levels and higher secretion of IL-10. Furthermore, oral administration of either live F. prausnitzii or its supernatant markedly reduced the severity of TNBS colitis and tended to correct the dysbiosis associated with TNBS colitis (Sokol, et al., 2008). Moreover, F. prausnitzii was suggested to induce a similar interleukin 10 (IL-10)-producing regulatory T cell population in humans (Sarrabayrouse, et al., 2014). The FP A2-165 reference strain significantly decreased colonic hypersensitivity induced by either NMS in mice or partial restraint stress in rats (Miquel, et al., 2016). Faecalibacterium prausnitzii is strongly associated with Atopic Dermatitis (AD). A recent study showed that the fecal samples from patients with AD showed decreased levels of butyrate and propionate, which have anti-inflammatory effects (Song, et al., 2016).

F. prausnitzii may also be involved in reduction of colitis through in vivo modulation of metabolites. Previous studies demonstrated that metabolites of F. prausnitzii could affect gut physiology and immunity. The supernatant from F. prausnitzii cultures significantly reduced IL-8 secretion induced by IL-10 (Sokol, et al., 2008). Interestingly, Sokol, et al., 2008 also examined the antimicrobial effects of F. prausnitzii supernatant and found no such effect in vitro. Moreover, salicylic acid was found directly involved in the protective effect of F. prausnitzii (Miquel, et al., 2015). Additionally, after treatment with F. prausnitzii supernatant, the plasma levels of IL-17A and IL-6 (P<0.05), the protein and mRNA expression of IL-17A and RORγt, and the Th17 cell ratio of spleen cells (P<0.01) were significantly decreased compared to the model group (Huang, et al., 2016).

A recent study demonstrated that a peptide, identified in the F. prausnitzii culture supernatants, has an anti-inflammatory effect (Quevrain, et al., 2016). Two fractions (F2 and F3) from F. prausnitzii supernatant exerted inhibitory effects on IL1-β-induced IL-8 secretion in intestinal epithelial CaCO2 cells. Interestingly, the seven peptides derived from a single microbial anti-inflammatory molecule (MAM), a protein of 15 kDa, and comprising 53% of non-polar residues. Transfection of MAM cDNA in epithelial cells led to a significant decrease in the activation of the nuclear factor (NF)-κB pathway with a dose-dependent effect (Quevrain, et al., 2016). They also observed that anti-inflammatory effect was not abrogated by heat (above 70° C.), enzyme digestion (trypsin, lipase, amylase) or MW filtration acid and HBTU/DIEA for the activation. The crude peptides were purified by HPLC to obtain purity over 97% (Quevrain, et al., 2016). These results indicated that various metabolites other than MAM could contribute to this effect.

Short-chain fatty acids (SCFA) such as acetate, n-propionate, n-butyrate are end products of bacterial anaerobic fermentation of dietary fiber and are likely candidates for regulating immune response in the intestines (Chang, et al., 2014). SCFA can be found at high concentrations in the large intestine (e.g., 20 mM n-butyrate in colonic lumen) (Louis, et al., 2009). Chang et al demonstrated that the n-butyrate can modulate the function of intestinal macrophages. Treatment of macrophages with n-butyrate led to the down-regulation of lipopolysaccharide-induced proinflammatory mediators, including nitric oxide, IL-6 and IL-12, but did not affect levels of TNF-α or MCP-1 (Chang, et al., 2014).

The anti-inflammatory effect exerted by F. prausnitzii was associated with various metabolic changes but the precise molecular mechanisms remained undefined. Targeting its metabolic pathways could be an attractive therapeutic strategy in IBD (Miquel, et al., 2015).

While these anti-inflammatory effects have been described, there has been no previous disclosure that F. prausnitzii compositions have antibacterial activity. Experiments described herein demonstrate that F. prausnitzii compositions have antibacterial activity, and for example, inhibit the growth of or kill bacteria such as Escherichia coli, Klebsiella pneumoniae and Staphylococcus aureus.

Accordingly, the present invention provides methods of using Faecalibacterium spp. and compositions derived from cultures of Faecalibacterium spp. to prevent or decrease growth of other microorganisms, particularly pathogenic organisms. The F. prausnitzii compositions of the present invention may comprise live or dead strain(s) of F. prausnitzii or may be F. prausnitzii supernatants obtained from culturing F. prausnitzii. In some preferred embodiments, the F. prausnitzii used in the compositions and methods of the present invention is isolated as described in Foditsch et al., PLOS ONE|DOI:10.1371/journal.pone.0116465(2014), incorporated herein by reference in its entirety. It will be understood to those of ordinary skill in the art that suitable strains of F. prausnitzii may be obtained as described in Foditsch (2014) and screened as described herein for antimicrobial activity. Thus, the current invention is not limited to the specific strains described herein. Multiple strains with antimicrobial activity are described in the examples and additional strains for use in the methods and compositions of the present invention can be obtained as described herein.

In some embodiments, compositions and formulations of the present invention comprise an effective amount of a F. prausnitzii composition, for example, one or more live or dead F. prausnitzii strains (or combinations thereof) or a F. prausnitzii culture supernatant. In some embodiments, the effective amount is an amount sufficient to inhibit the growth of a target microorganism. In some embodiments, the effective amount is an amount sufficient to prevent the growth of a target microorganism. In some embodiments, the effective amount is an amount sufficient to kill the target microorganism. In some embodiments, the amount is effective to reduce the amount of the microorganism in the subject by at least 99%, 90%, 80%, 70%, 60% or 50% as compared to the amount of microorganism present prior to administration. The amount of the microorganism present can be determined, for example, by culturing a sample or swab taken from a subject that is being treated. In some embodiments, the amount is effective to treat or prevent an infection by the target microorganism.

### I. Compositions and Kits

In some embodiments, the present invention provides F. prausnitzii compositions and kits. In some embodiments, F. prausnitzii compositions comprise one or more live and/or dead Faecalibacterium spp. or strains. The present invention is not limited to a particular Faecalibacterium spp. or strain. Examples include, but are not limited to, Faecalibacterium prausnitzii, and in particularly preferred embodiments include strains 24, 30, 266 and 267.

In some embodiments, compositions comprise one or more additional components (e.g., including but not limited to, additional additive selected from the group consisting of an energy substrate, a mineral, a vitamin, or combinations thereof).

In some embodiments, compositions comprise one or more (e.g., 2 or more, 5 or more, 10 or more, etc.) additional strains of bacteria or other microorganisms (e.g., probiotic microorganisms). Examples include, but are not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, or combinations thereof. In some embodiments, multiple strains of the same bacteria are utilized in combination.

In some embodiments, bacteria are live cells or freeze-dried cells. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze-dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. The viability of freeze-dried bacteria in oil is maintained for at least nine months. Optionally live cells can be added to one of the above oils and stored.

In some embodiments, the F. prausnitzii compositions are supernatants from a culture of F. prausnitzii. The supernatants resulting from the culture of F. prausnitzii may preferably be dried, for example by spray drying, vacuum drying, freeze-drying or lyophilization. The resulting powder may preferably be formulated as a powder, such as a dispersible powder, bolus, gel, liquid drench, capsule, or paste

In some embodiments, the F. prausnitzii compositions are part of a milk replacer (e.g., for administration to a neonatal or young animal). In some embodiments, compositions comprise one or more probiotic bacteria as described herein in combination with a milk protein (e.g., caseins or whey proteins).

In some embodiments, F. prausnitzii compositions are added to nutraceuticals, food products, or foods. In some embodiments, to give the composition or nutraceutical a pleasant taste, flavoring substances such as for example mints, fruit juices, licorice, Stevia rebaudiana, steviosides or other calorie free sweeteners, rebaudioside A, essential oils like eucalyptus oil, or menthol can optionally be included in compositions of embodiments of the present invention.

In some embodiments, F. prausnitzii compositions are formulated in pharmaceutical compositions. The bacteria of embodiments of the invention may be administered alone or in combination with pharmaceutically acceptable carriers or diluents, and such administration may be carried out in single or multiple doses.

Compositions may, for example, be in the form of tablets, resolvable tablets, capsules, bolus, drench, pastes, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the invention may be in a form suitable for oral, topical, buccal administration. Depending upon the disorder and subject to be treated and the route of administration, the compositions may be administered at varying doses.

For oral or buccal administration, bacteria of embodiments of the present invention may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavoring, preservative, sweetening or coloring agents. Diluents such as water, ethanol, propylene glycol, glycerin and combinations thereof may also be included.

Other suitable fillers, binders, disintegrants, lubricants and additional excipients are well known to a person skilled in the art.

In some embodiments, bacteria are spray-dried. In other embodiments, bacteria are suspended in an oil phase and are encased by at least one protective layer, which is water-soluble (water-soluble derivatives of cellulose or starch, gums or pectins; See e.g., EP 0 180 743, herein incorporated by reference in its entirety).

In some embodiments, the present invention provides kits, pharmaceutical compositions, or other delivery systems for use in preventing or inhibiting growth of a target microorganism in a subject. The kit may include any and all components necessary, useful or sufficient for research or therapeutic uses including, but not limited to, one or more probiotic bacteria or supernatant compositions, pharmaceutical carriers, and additional components useful, necessary or sufficient for improving weight gain, providing prophylaxis against diarrhea and/or improving feed efficiency in an animal. In some embodiments, the kits provide a sub-set of the required components, wherein it is expected that the user will supply the remaining components. In some embodiments, the kits comprise two or more separate containers wherein each container houses a subset of the components to be delivered.

Optionally, compositions and kits comprise other active components in order to achieve desired therapeutic effects.

### II. Therapeutic and Supplement Uses

Embodiments of the present invention provide Faecalibacterium spp. compositions (e.g., F. prausnitzii compositions as described above alone or in combination with additional probiotic bacteria) for use in inhibiting or preventing growth of a microorganism in a subject, or for killing a microorganism in a subject, or for treating or preventing an infection by a microorganism in a subject. In preferred embodiments, an effective amount of the F. prausnitzii composition or formulation is administered to the subject under conditions that growth of a target microorganism such as Escherichia coli, Klebsiella pneumoniae or Staphylococcus aureus is prevented or inhibited, or so that the microorganism is killed.

### EXPERIMENTAL

The following examples are provided in order to demonstrate and further illustrate certain preferred embodiments and aspects of the present invention and are not to be construed as limiting the scope thereof.

### Example 1

The F. prausnitzii strains used in the following examples were isolated as described in Foditsch et al., PLOS ONE|DOI:10.1371/journal.pone.0116465(2014). Briefly, fecal samples were collected from 7-28 days-old healthy Holstein calves and from 10-30 days-old healthy piglets. The samples were gently collected from the rectum and immediately placed in a tube containing 12 ml of VTR2RF broth. The tubes were sealed and transported until further processing. The subsequent procedures

were performed in an anaerobic chamber (BacBasic chamber, Sheldon Manufacturing, Inc., Cornellius, Oreg.). All samples were serially diluted in Anaerobic Dilution Blank (Anaerobe Systems, USA) and plated on VTR2RF agar.
After 48 h, about 10 typical colonies from each sample were selected and single-colony purified in VTR2RF agar. The isolates were stored at 280° C. in VTR2RF broth containing 16% of glycerol.

The VTR2RF media used as transport, enrichment, and isolation media was composed of the anaerobic media Versa TREK REDOX 2 (Trek Diagnostic Systems, Cleveland—Ohio) supplemented with 30% filtered rumen fluid. Rumen fluid was collected from fistulated cows, centrifuged at 12,0006 g for 30 min, the supernatant was filter-sterilized (Corning Incorporated Life Sciences, Tewksbury—Mass.) 3 times and stored at 4° C. VTR2RF agar was additionally supplemented with 0.5% (w/v) yeast extract (BD, Franklin Lakes, N.J.), 5 mg/l (w/v) hemin (Sigma-Aldrich, St. Louis, Mo.), 1 mg/l (w/v) cellobiose (Sigma-Aldrich), 1 mg/ml (w/v) maltose (Sigma-Aldrich), and 0.5 mg/ml (w/v) L-cystein (Sigma-Aldrich).

### Example 2

1. Screening of Significant F. prausnitzii Strains

To investigate if F. prausnitzii supernatant (SN) could inhibit the growth of E. coli, a batch of F. prausnitzii strains were selected to carry out the growth inhibition test, which included 15 strains (strain 4, 24, 24D, 27, 30, 32, 34, 36D, 58, 69D, 74, 266, 267, 272X and 297X). F. prausnitzii A2-165 (DSM 17677) was used as a control strain. The enterotoxigenic Escherichia coli ATCC 31616 (K19), known to induce diarrhea in animals, was used as the E. coli treatment strain.

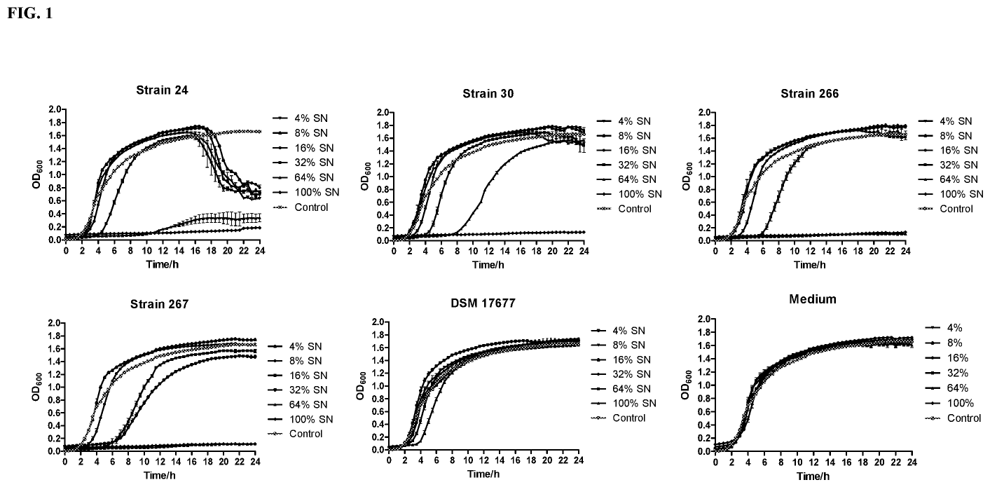

Results showed that the supernatant of F. prausnitzii strains 24, 30, 266 and 267 displayed a significant inhibition effect on the growth of Escherichia coli ATCC 31616 (FIG. 1). There was no significant effect by adding the supernatant of F. prausnitzii DSM 17677 or the medium for the growth of F. prausnitzii strains. Moreover, the inhibition effect increased with increases in the percentage of F. prausnitzii supernatant, which indicates that the inhibition effect is dose-dependent. Interestingly, the growth of E. coli ATCC 31616 was completely suppressed by adding 64% or 100% supernatant of F. prausnitzii strain 266 and strain 267 into the LB broth for the growth of E. coli ATCC 31616. Additionally, for the supernatant of F. prausnitzii strain 24, it caused a significant decrease in the number of E. coli ATCC 31616 after the stationary phase, which indicates that the supernatant of F. prausnitzii strain 24 kills E. coli ATCC 31616.

The supernatant of F. prausnitzii strains 24, 30, 266 and 267 had significant inhibition effect on the growth of E. coli ATCC 31616, and was selected for further study.

2. The Supernatant of F. prausnitzii Displayed Widespread Inhibition Effect on E. coli

In order to explore if the inhibition effect of F. prausnitzii supernatant is specific to E. coli ATCC 31616, we investigated the effect of the supernatant of F. prausnitzii strains 24, 30, 266 and 267 on the growth of E. coli ATCC 25922, a non-pathogenic E. coli reference strain.

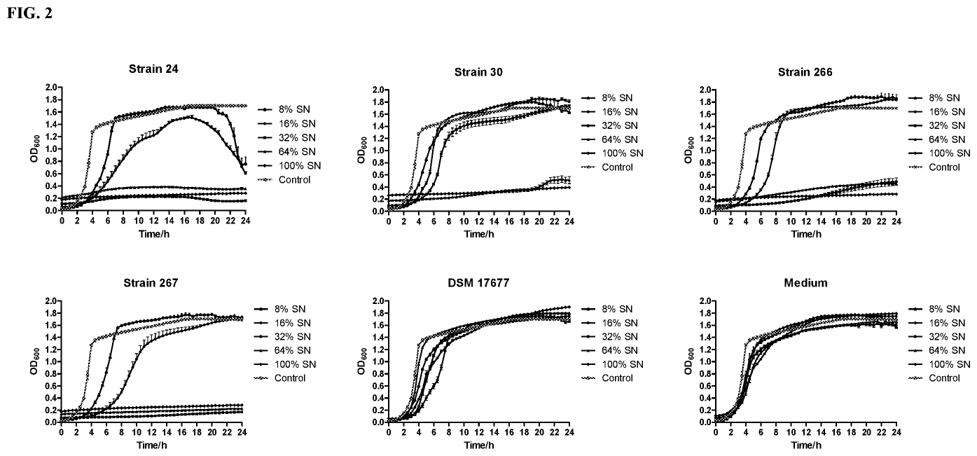

Significant inhibition on the growth of E. coli ATCC 25922 was observed, which displayed a dose-dependent manner (FIG. 2). In the same way, the growth of E. coli ATCC 25922 was completely suppressed by adding >32% supernatant of F. prausnitzii strains into the LB broth for the growth of E. coli. These results show that the inhibition effect of F. prausnitzii supernatant is universal for various E. coli strains.

3. The Supernatant of F. prausnitzii Displayed Significant Inhibition Effect on Klebsiella pneumonia

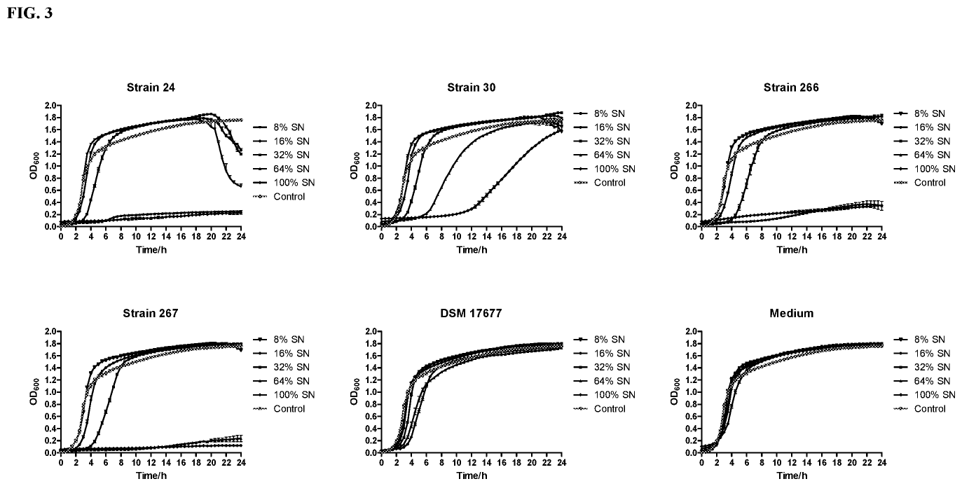

In order to explore if the inhibition effect of F. prausnitzii supernatant is specific to E. coli, we carried out the growth inhibition test on Klebsiella pneumoniae strain 22326, a pathogenic Klebsiella pneumoniae strain that induces mastitis in dairy cows. Results showed that the supernatant of F. prausnitzii strain 24, 30, 266 and 267 has significant inhibition effects on the growth of Klebsiella pneumoniae strain 22326 in a dose-dependent manner (FIG. 3). Similarly, the OD₆₀₀ of Klebsiella pneumoniae decreased significantly by adding the supernatant of F. prausnitzii strain 24 after 18 h incubation.

4. The Supernatant of F. prausnitzii Displayed Significant Inhibition Effect on Staphylococcus aureus

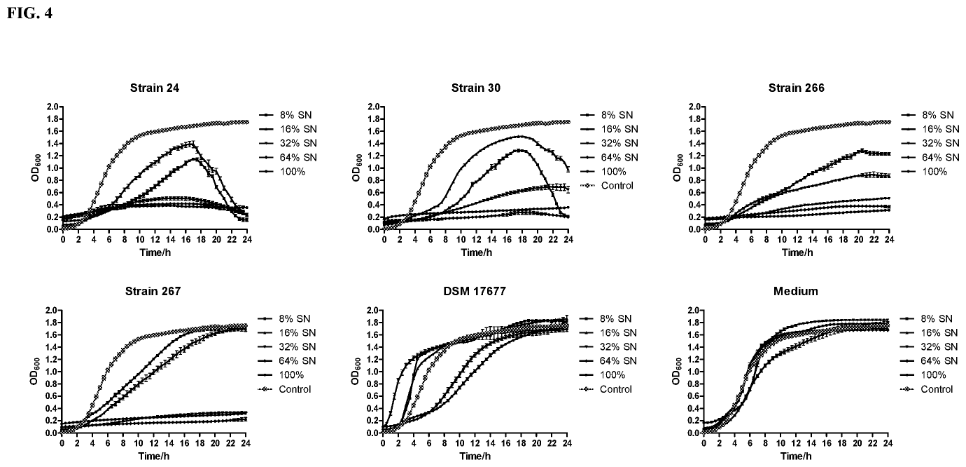

In order to explore if the inhibition effect of F. prausnitzii supernatant is specific to Gram negative bacteria, growth inhibition studies were carried out using Staphylococcus aureus ATCC 27708. Results showed that the supernatant from all four strains of F. prausnitzii had significant inhibition effects on the growth of Staphylococcus aureus ATCC 27708 in a dose-dependent manner. Similarly, the OD₆₀₀ of Staphylococcus aureus ATCC 27708 decreased significantly by adding the supernatant of F. prausnitzii strain 24 after 18 h incubation (FIG. 4).

5. The Supernatant of F. prausnitzii Displayed Significant Inhibition Effect on Methicillin-Resistant Staphylococcus aureus (MRSA)

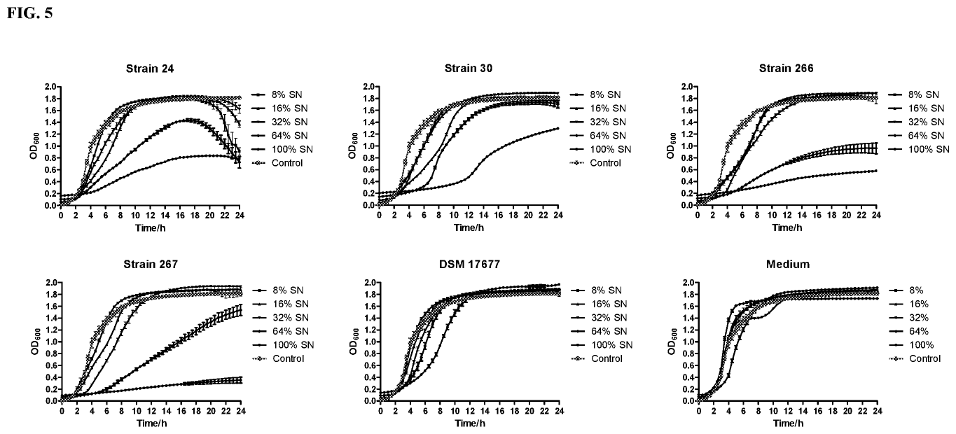

MRSA is a serious type of S. aureus bacteria that is resistant to many different kinds of antibiotics. In order to explore if the inhibition effect of F. prausnitzii supernatant is applicative to MRSA, MRSA 4454 was selected to do the growth inhibition tests. Results showed that supernatants from F. prausnitzii strains 24, 30, 266 and 267 have significant inhibition effect on the growth of MRSA in a dose-dependent manner. Similarly, the OD₆₀₀ of MRSA cell decreased significantly by adding the supernatant of F. prausnitzii strain 24 after 18 h incubation (FIG. 5).

### Example 3

1. The Supernatant of F. prausnitzii Strain 24 Kills E. coli ATCC 31616

In our previous study, we demonstrated that the number of E. coli ATCC 31616 (K19) decreased after 16 h incubation with the supernatant of F. prausnitzii strain 24. Then we further explored if the supernatant of F. prausnitzii strain 24 could kill E. coli ATCC 31616.

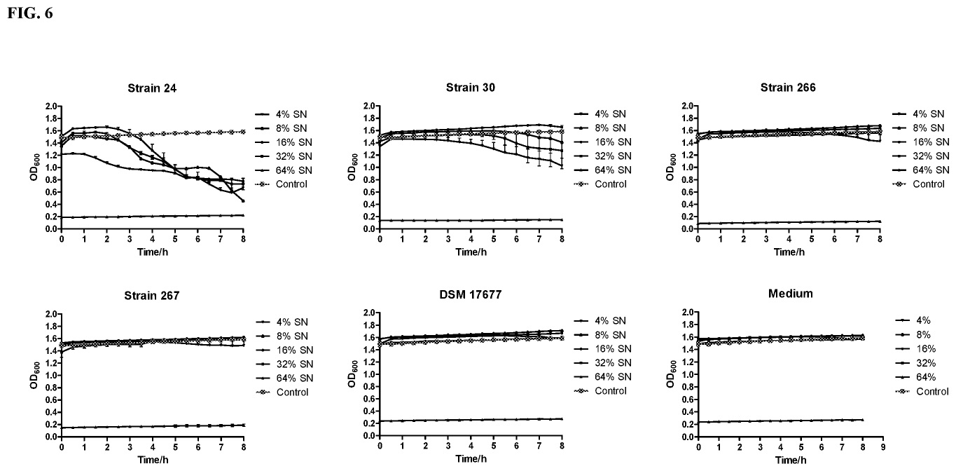

E. coli ATCC 31616 was recovered after reaching the stationary phase. The OD₆₀₀ of culture by adding F. prausnitzii supernatant into the cell were detected every half an hour until 8 h. Results showed that the OD₆₀₀ of culture decreased significantly for the group of F. prausnitzii strain 24 (FIG. 6). This result agreed with the previous study that the number of E. coli ATCC 31616 reduced after 16 h incubation with the supernatant of F. prausnitzii strain 24.

2. The Supernatant of F. prausnitzii Strain 24 Significantly Decreased the Number of Different Bacteria

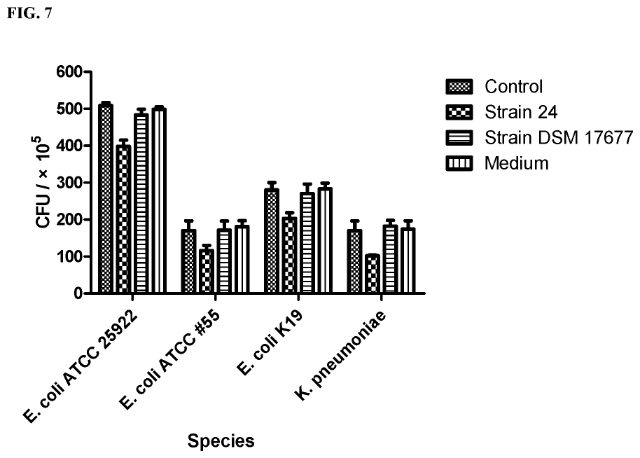

To verify our hypothesis, we further carried out CFU counting tests after the co-incubation of various bacteria with the supernatant of F. prausnitzii strain 24. A total of 4 different kinds of bacteria were selected to do this experiment, including a common non-pathogenic E. coli reference strain E. coli ATCC 25922, two important pathogenic E. coli reference strains E. coli ATCC 31616 (K19) and E. coli ATCC #55, and a pathogenic Klebsiella pneumoniae that induces mastitis in dairy cows, K. pneumoniae strain 22326. The cell of various bacteria were recovered after reaching the stationary phase. Then the cell of bacteria was co-incubated with the supernatant of F. prausnitzii strain 24. The mixtures of supernatant and E. coli were taken at 60 min and serially diluted. These dilutions were plated on LB agar and incubated overnight to determine cell viability and number of CFU. The results showed that the supernatant of F. prausnitzii strain 24 maintained its inhibitory activity to all the test strains (FIG. 7). Compared with the control group, supernatant of F. prausnitzii strain DSM 17677 and culture medium, a progressive reduction in bacterial population was observed after co-incubation with the supernatant of F. prausnitzii strain 24.

### REFERENCES

Chang P V, Hao L, Offermanns S, Medzhitov R. The microbial metabolite butyrate regulates intestinal macrophage function via histone deacetylase inhibition. Proc Natl Acad Sci USA 2014; 111:2247-2252.
Homef M W, Pabst O. Real friends: Faecalibacterium prausnitzii supports mucosal immune homeostasis. Gut 2016; 65:365-367.
Huang X L, Zhang X, Fei X Y, Chen Z G, Hao Y P, Zhang S, Zhang M M, Yu Y Q, Yu C G. Faecalibacterium prausnitzii supernatant ameliorates dextran sulfate sodium induced colitis by regulating Th17 cell differentiation. World J Gastroenterol 2016; 22:5201-5210.
Louis P, Flint H J. Diversity, metabolism and microbial ecology of butyrate-producing bacteria from the human large intestine. FEMS Microbiol Lett 2009; 294:1-8.
Miquel S, Leclerc M, Martin R, Chain F, Lenoir M, Raguideau S, Hudault S, Bridonneau C, Northen T, Bowen B, Bermudez-Humaran L G, Sokol H, Thomas M, Langella P. Identification of metabolic signatures linked to anti-inflammatory effects of Faecalibacterium prausnitzii. MBio 2015; 6.
Miquel S, Martin R, Lashermes A, Gillet M, Meleine M, Gelot A, Eschalier A, Ardid D, Bermudez-Humaran L G, Sokol H, Thomas M, Theodorou V, Langella P, Carvalho F A. Anti-nociceptive effect of Faecalibacterium prausnitzii in non-inflammatory IBS-like models. Sci Rep 2016; 6:19399.
Miquel S, Martin R, Rossi O, Bermudez-Humaran L G, Chatel J M, Sokol H, Thomas M, Wells J M, Langella P. Faecalibacterium prausnitzii and human intestinal health. Curr Opin Microbiol 2013; 16:255-261.
Quevrain E, Maubert M A, Michon C, Chain F, Marquant R, Tailhades J, Miquel S, Carlier L, Bermudez-Humaran L G, Pigneur B, Lequin O, Kharrat P, Thomas G, Rainteau D, Aubry C, Breyner N, Afonso C, Lavielle S, Grill J P, Chassaing G, Chatel J M, Trugnan G, Xavier R, Langella P, Sokol H, Seksik P. Identification of an anti-inflammatory protein from Faecalibacterium prausnitzii, a commensal bacterium deficient in Crohn's disease. Gut 2016; 65:415-425.
Sarrabayrouse G, Bossard C, Chauvin J M, Jarry A, Meurette G, Quevrain E, Bridonneau C, Preisser L, Asehnoune K, Labarriere N, Altare F, Sokol H, Jotereau F. CD4CD8alphaalpha lymphocytes, a novel human regulatory T cell subset induced by colonic bacteria and deficient in patients with inflammatory bowel disease. PLoS Biol 2014; 12:e1001833.
Segain J P, Raingeard de la Bletiere D, Bourreille A, Leray V, Gervois N, Rosales C, Ferrier L, Bonnet C, Blottiere H M, Galmiche J P. Butyrate inhibits inflammatory responses through NFkappaB inhibition: implications for Crohn's disease. Gut 2000; 47:397-403.
Sokol H, Pigneur B, Watterlot L, Lakhdari O, Bermudez-Humaran L G, Gratadoux J J, Blugeon S, Bridonneau C, Furet J P, Corthier G, Grangette C, Vasquez N, Pochart P, Trugnan G, Thomas G, Blottiere H M, Dore J, Marteau P, Seksik P, Langella P. Faecalibacterium prausnitzii is an anti-inflammatory commensal bacterium identified by gut microbiota analysis of Crohn disease patients. Proc Natl Acad Sci USA 2008; 105:16731-16736.
Song H, Yoo Y, Hwang J, Na Y C, Kim H S. Faecalibacterium prausnitzii subspecies-level dysbiosis in the human gut microbiome underlying atopic dermatitis. J Allergy Clin Immunol 2016; 137:852-860.
Winter S E, Winter M G, Xavier M N, Thiennimitr P, Poon V, Keestra A M, Laughlin R C, Gomez G, Wu J, Lawhon S D, Popova I E, Parikh S J, Adams L G, Tsolis R M, Stewart V J, Baumler A J. Host-derived nitrate boosts growth of E. coli in the inflamed gut. Science 2013; 339:708-711.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of preventing or inhibiting growth of a target microorganism in a subject in need thereof comprising administering to the subject an effective amount of a Faecalibacterium spp. composition.

2. The method of claim 1, wherein the Faecalibacterium spp. is Faecalibacterium prausnitzii.

3. The method of claim 1, wherein said Faecalibacterium spp. has antimicrobial activity.

4. The method of claim 3, wherein said effective amount of Faecalibacterium spp. prevents, inhibits the growth of, or kills a bacteria selected from the group consisting of Escherichia coli, Klebsiella pneumonia, Staphylococcus aureus, and combinations thereof.

5. The method of claim 4, wherein said Escherichia coli is Escherichia coli ATCC 31616 (K19).

6. The method of claim 1, wherein the Faecalibacterium spp. composition comprises an effective amount of dead or inactivated Faecalibacterium spp.

7. The method of claim 5, wherein the Faecalibacterium spp. composition is formulated powder, bolus, gel, liquid drench, capsule, or paste.

8. The method of claim 7, wherein the Faecalibacterium spp. composition further comprises a bulking agent.

9. The method of claim 8, wherein the bulking agent is selected form the group consisting of milk powder, skim milk powder, corn starch, corn meal, and soybean meal.

10. The method of claim 1, wherein the Faecalibacterium spp. composition is a Faecalibacterium spp. culture supernatant from a culture of the Faecalibacterium spp.

11. The method of claim 10, wherein the Faecalibacterium spp. composition is formulated powder, bolus, gel, liquid drench, capsule, or paste.

12. The method of claim 11, wherein the Faecalibacterium spp. composition further comprises a bulking agent.

13. The method of claim 12, wherein the bulking agent is selected form the group consisting of milk powder, skim milk powder, corn starch, corn meal, and soybean meal.

14. The method of claim 1, wherein the composition is coadministered with at least a second probiotic organism selected from the group consisting of Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, and combinations thereof.

15. The method of claim 1 wherein the composition is formulated with an additional additive selected from the group consisting of an energy substrate, a mineral, a vitamin, and combinations thereof.

16. The method of claim 1, wherein the target microorganism is a pathogenic microorganism.

17. The method of claim 1, wherein the target microorganism is selected from the group consisting of Escherichia coli, Klebsiella pneumoniae and Staphylococcus aureus.

18. The method of claim 1, wherein the subject is a domestic animal.

19. The method of claim 18, wherein the domestic animal is selected from the group consisting of cattle, sheep, swine, and horses.

20. The method of claim 19, wherein the animal is a calf.

21. The method of claim 1, wherein the amount is effective to kill the target microorganism.

22. The method of claim 1, wherein the amount is effective to reduce the amount of the microorganism in the organism by at least 50% as compared to the amount of microorganism present prior to administration.

23-47. (canceled)
